(19) United States
(12) Patent Application Publication
(10) Pub. No.: US 2025/0281316 A1
(43) Pub. Date: Sep. 11, 2025

(54) Back Brace

(71) Applicant: Icarus Medical, LLC, Charlottesville, VA (US)

(72) Inventors: David T. Johnson, Charlottesville, VA (US); Evan Eckersley, Charlottesville, VA (US); Philip Miller, Charlottesville, VA (US)

(21) Appl. No.: 19/074,263

(22) Filed: Mar. 7, 2025

Related U.S. Application Data
(60) Provisional application No. 63/562,351, filed on Mar. 7, 2024.

(51) Int. Cl. A61F 5/02

(52) U.S. Cl. CPC A61F 5/024; A61F 5/022; A61F 5/028

(57) ABSTRACT

A novel orthosis for correcting and modifying the position of vertebrae in the spine and neck. A plurality of force vectors are generated by the orthosis which transfers the forces to the wearer to counter compression and/or displacement of the vertebrae. In addition, the magnitude of the force vectors can be modified while wearing the orthosis which improves comfort and efficacy.

### CROSS-REFERENCE TO RELATED APPLICATION

The present application relies on the disclosures of and claims priority to and the benefit of the filing date of U.S. Patent Application No. 63/562,351, filed Mar. 7, 2024. The disclosures of that application are hereby incorporated by reference herein in their entireties.

### BACKGROUND

The spine is one of the most vital aspects to both structure and stability in the human body. It is heavily innervated, utilizes a vast network of tendons and ligaments, and supports the slow twitch muscular system that enables postural control. All of these factors considered, the back is an extremely common location for injuries and chronic conditions. These complexities create further difficulties in diagnosis and treatment options. MRI's and CAT scans conducted for back pain are often inconclusive. This results in wasted time and money, while also increasing patients' frustrations. Those who are convinced to schedule surgical procedures are met with further complications and poor outcomes. Currently in the U.S. an estimate of around 50% of surgical operations conducted on the spine are considered unnecessary. Back bracing was created as a way to delay or eliminate the need for any prescribed testing or procedures. These braces provide postural stability and strength while alleviating pressure on the lower back, and often help patients find relief for both acute and chronic conditions.

Bracing devices are typically worn all day in high stress environments, and as such require high comfortability paired with discrete design qualities. Comfort, being one of the most sought after factors in choosing a product, can be difficult to achieve. Simply increasing the padding is not a viable option as this subsequently increases the braces bulk. Similarly, decreasing the size of the brace can significantly reduce patient comfort and brace compliance. Since most braces are restricted by only one of the above requirements, patients will often forgo use of the product in long term circumstances, further exacerbating the injuries to their back. Thus, there is a need for a brace that provides personalized, dynamic and functional support while maintaining a slim profile and maximizing comfort.

Contemporary brace procedure requires little to no custom fabrication which can lead to ineffective bracing. These deficiencies primarily include one-size-fits-all posterior panels. Often, posterior brace panels do not completely conform to the wearer's back, causing discomfort and brace migration to ineffective positions. The widely accepted S-shaped curve that is considered the spine becomes unduly restricted through the application of a linear posterior panel. In general, brace wearers' spinal columns can range significantly in shape, strength and need of support. Additionally, patient specific information including their clinical indication and location of condition within the spine is not taken into account. Thus there is a need for a brace in which the posterior panel is specifically manufactured from a detailed mapping of the patient's natural undulations.

Standardized products are further limited in postural support due to the one-size-fits all mentality. The braces are created with the capability to be worn by any patient regardless of their individual diagnosis and brace requirements. By conforming to the patient's body they are simply adapting the braces shape, without the ability to control its level of support. This ensures that pressure is evenly applied throughout the posterior panel, regardless of individual specification. The only adjustability in force magnitude comes from removal and replacement of the posterior panel, and changing its material to alter mechanical properties. Furthermore, it is well understood that a lack of brace functionality, due to poor fitment or improper strength assistance, can lead to atrophy among the posterior muscles, ultimately creating a patient dependency upon the brace. Targeting and gradually reducing the assisting force over the course of an injury can create a method in which the back is able to regenerate strength in a healthy painless manner. Thus there is a need for a brace that allows for patient directed force magnitude adjustment, in order to comply with the patient's natural curvature, postural needs, and level of support.

In many cases, regardless of the cause, back pain can be alleviated by reducing inter-vertebral pressure at the site of an injury. Traction braces for the back and neck are designed to reduce the compression of the spine by applying a “distraction force” or “joint distraction”-that is, a force applied to the vertebrae of the spine. Typical traction braces only apply a distraction force along the local long axis of the spine. What is needed is a more generalized orthotic where, in cases, multiple forces can be applied such that the resultant force vector (net force vector) produces a decompressing force that can be moved/positioned to specific zones or points along the spine.

Scoliosis represents another major clinical indication for which back braces are prescribed. However, as in the above example of therapeutic braces, scoliosis braces present significant limitations in customized and functional support.

Scoliosis, a condition characterized by an abnormal lateral curvature of the spine, can have various impacts on individuals and their quality of life. Scoliosis can cause physical discomfort and pain, particularly in the back, shoulders, and neck. This discomfort may range from mild to severe, depending on the severity of the curvature and whether it progresses over time. Severe scoliosis can restrict movement and flexibility, impacting an individual's ability to perform daily activities, participate in physical exercise, or engage in certain occupations or hobbies. Additionally, the visible asymmetry of the spine and torso caused by scoliosis may affect body image and self-esteem in the affected, young population. Current braces exacerbate this issue due to their lack of style and low-profile design. This, in turn, presents a burden in terms of stress, damage to social life, and financial burden.

Overall, the impact of scoliosis varies depending on factors such as the severity of the curvature, age of onset, effectiveness of treatment, and individual resilience. Therefore, an effective solution requires a high degree of personalization and adjustable functionality in order to meet each patients' specific needs and lifestyle.

The treatment methods for scoliosis depend on various factors, including the severity of the curvature, the age of the patient, and the likelihood of progression. Bracing is often recommended for adolescents with moderate scoliosis (curvature between 25-40 degrees) who are still growing. The brace helps to prevent further progression of the curvature by applying pressure to the spine. The specific type of brace and duration of wear depend on the individual's condition and growth potential.

Bracing solutions specifically face multiple limitations including inconclusive and variable effectiveness, poor compliance due to lack of comfort, appearance or inconvenience. The effectiveness of treatments such as bracing and physical therapy can vary depending on factors such as the severity and progression of the curvature, the age of the patient, and compliance with treatment protocols. Not all patients respond equally to these conservative measures. Furthermore, there exist no bracing solutions that offer easily adjustable corrective force after production. Being able to control the specific location throughout the spine with a desired magnitude of force would greatly ease the patient experience and comfortability.

A variety of back braces solutions are known in the prior art, but the current invention described herein is an improvement to each and every one of them and the industry as a whole.

U.S. Pat. No. 10,517,749 assigned to DJO, LLC describes a simple band that can be tightened around the abdomen of the wearer. The brace contains multiple panels that are connected via lace, which when tensioned, compresses inward to fit snugly no matter the body shape. Compression of the abdomen further creates intracavitary pressure which is known to alleviate some forms of back pain. This patent cites a rotary tension device to incrementally adjust the lace tension based on user preference, and then release all compression in preparation for removal. While effective at reducing pain, this system results in no permanent physiological changes that solve the cause of the problem: e.g., correcting the position of the vertebrae in the spine.

U.S. Pat. No. 9,393,149 assigned to Aspen Medical Products, Inc. describes a double pull body brace system that allows the wearer to adjust the level of tension and support by pulling on two tabs to cinch the brace together. This applies a compressive force which alleviates pain. The device consists of one panel that wraps around the wearer's abdomen and attaches anteriorly via a hook-and-loop fastener to the patient's waist. While this brace is form fitting and reduces pain, it does not result in any permanent physiological changes to address the root cause of the problem.

U.S. Pat. No. 10,117,770 assigned to Fiji Manufacturing, LLC describes an adjustable lumbar brace. This device consists of a plurality of panels that use a tensioning system to adjust the level of compression and support in the lumbar region. While the tension system can be increased or decreased to the wearer's desired level of support, it also acts as a way to contour the brace the shape of the spine. Increasing the tension bends the lumbar curve to fit snugly along the posterior of the spine. This curvature allows the brace to fit more comfortably and ultimately mitigate migration, but it does not result in any permanent physiological changes to address the root cause of the problem.

U.S. Pat. No. 6,213,968 assigned to BioCybernetics International describes a custom fitted orthotic device consisting of a lace tension system that tightens the brace to the wearer's body. It includes multiple modes of adjustment to asymmetrically configure the brace which is claimed to increase patient compliance. Introducing multiple adjustment mechanisms can better fit the wearer, reduce brace migration, and have better immobilization control. Ultimately, the device does not solve the problem that is causing the back pain and acts only to alleviate pain by applying compressive pressure.

U.S. Pat. No. 8,920,353 assigned to Orthotic Solutions, LLC is an adjustable lumbo-sacral orthosis that consists of a tension system to adjust the fit of the orthosis around the patient's abdomen, as well as tightens an independently movable component that bends to the shape of their lumbar region. The adjustable lumbar panel is intended to ensure proper alignment throughout daily activities and prevent migration during transitions from sitting to standing. However, it does not appear to provide any spine elongation or provide permanent correction.

The Airy™ Brace is an adjustable orthotic designed to treat scoliosis. This device consists of a system of adjustable panels that are connected through a rigid vertical support structure. The panels are off the shelf sized and can be adjusted in height, angle, and compression. Featuring an elastic band, the panels are compressed together to apply a corrective force and adjust the spinal cord positioning. While the device can be adjusted to accommodate growth of the wearer, the adjustments cannot be made while wearing the device.

With the exception of the Airy™ Brace, the prior art uses adjustability to improve fit and comfort. What is needed is the ability to easily modify a plurality of vector forces—both the direction of the applied force and the magnitude—at a plurality of locations along the orthotic to permanently correct the positions of different vertebrae of the spine and neck.

### SUMMARY OF THE INVENTION

A range of mechanisms that can create and adjust distraction forces and/or adjustment forces are described herein, such as ramps, levers, screws, pulleys, and/or a force multiplying mechanism (such as a dial) acting on other bodies to generate the desired system of forces. The effectiveness of these devices can be enhanced by a well-fitting, conforming orthosis that places the action (or origin) of the distraction mechanism in the desired position. This effect can be further enhanced by using high-friction interface materials to “grip” the body of the patient. The change in circumference, for example, from the patient's hips to waist, creates a stable base from which to leverage push a distraction force. Other examples include changes in circumference or shape within the rib cage, changes from the rib cage to the under-arms, and within the neck between the shoulders and the cranium/mandible, when neck distraction is desired.

Back orthoses for the improved correction and unloading of the spine require a more advanced degree of personalization than is described in the prior art. Accordingly, the disclosed invention herein describes a user-adjustable brace that can be modified through various mechanisms sequentially in line with a regimen or as desired by the user.

Static or dynamic forces can be applied along or around all three axes of the spine depending on embodiment, including in the sagittal, coronal, and horizontal planes (see, FIG. 1).

Embodiments of the invention incorporate unique features to provide dynamic and personalized support to improve user outcomes. In embodiments, these features include 1) user control of the amount of corrective force with adjustable tensioning and compression systems, 2) user control of direction of corrective force, 3) an adjustment mechanism allowing the user to control the direction and magnitude of corrective force, 4) customization through 3D scan to 3D print processes, 5) modularity including incorporation of joints, hinges, brackets and tracks connecting rigid and/or semi-rigid components to each other or to the user/wearer and 6) optionally the incorporation of motors and sensors to yield a smart brace.

In general, the device may be used to improve outcomes for 1) spinal correction such as scoliosis bracing, 2) posture and support, 3) spinal distraction, decompression or unloading (described throughout as spinal distraction) 4) musculoskeletal strengthening and passive exoskeletons, and 5) post-operative applications.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention.

FIG. 1 is an illustration of the sagittal, coronal, and transversal planes of the human body.

FIG. 2 is a posterior view of a spine with scoliosis.

FIGS. 3a and 3b are posterior views of slidably connected body panel orthosis, illustrating the corrective forces applied to the back.

FIG. 4 is a posterior view of a multi-component back brace configured to treat scoliosis, featuring an adjustable tensioning component.

FIGS. 5a and 5b are posterior and anterior views of a multi-component back brace with an adjustable tension component.

FIGS. 6a and 6b are posterior and anterior views of a multi-component back brace with multiple adjustable tension components.

FIG. 7 is a posterior view of a conformable back brace to treat scoliosis.

FIG. 8 is a lateral view of a multi-component back brace to treat scoliosis.

FIG. 9 is a lateral view of a multi-component wedging back brace to treat scoliosis.

FIG. 10 is a posterior view of a traction back brace.

FIGS. 11a and 11b are posterior views of a wedging traction back brace.

FIGS. 12a and 12b are posterior views of a uniform and non-uniform wedging traction back brace.

FIG. 13 is an anterior view of a traction neck brace.

FIG. 14 is an anterior view of a wedging traction neck brace.

FIGS. 15a-15d show applications and mechanisms for a traction neck brace.

FIG. 16 is a posterior view of a sliding back brace to treat scoliosis, featuring a vertical beam and horizontal tracks.

FIG. 17 is a posterior view of a rack and pinion traction back brace.

### DETAILED DESCRIPTION

Orthoses for the spine need to be customized to the user's needs in multiple aspects: 1) adjustability in the amount, direction and location of applied force or forces within one or more anatomical axes, 2) a range of forces that are of a magnitude sufficient to correct or decompress the spine (preferably in a comfortable manner) and 3) a method in which the magnitude of support can be controllably lowered to regain musculoskeletal strength. Additionally, it is preferable to include 4) modularity to tune the brace to the specific indication, and 5) provide a custom-fit for the user's comfort, aesthetics and to optimize function and load distribution offer functional benefits.

In a similar manner, orthoses to treat scoliosis will benefit from the above features, especially modularity and adjustability. Scoliosis braces are currently replaced every six or so months (as the patient grows and their spine changes shape) presenting inconvenience to the user and adds to the high cost of treatment.

The disclosed invention provides the wearer with an adjustable brace that can be modified to follow a physiological regimen.

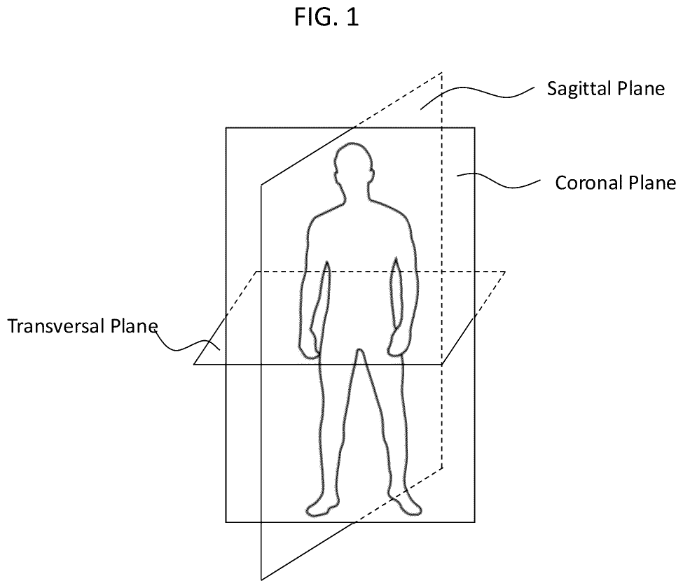

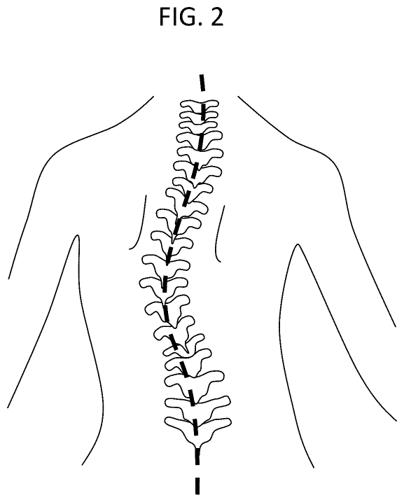

An example of a spine with scoliosis is shown in FIG. 2. As shown in FIG. 2 the curvature is most visible in the coronal plane, however in practice there may be curvature in any or all of the three body planes that are shown in FIG. 1.

Practitioners use physical examinations, X-rays, MRI scans, sonograms, and the like to create a representation of the curvature of the spine. A treatment plan is created by assessing the existing curvature and comparing them to the desired curvature. The treatment plan may include creating a substantially rigid, polymer shell for the patient to wear wherein the polymer shell applies a corrective force to the spine. As the spine moves towards the idealized position, a new polymer shell may be fabricated to continue the slow, subtle changes to the spinal position. In some cases, straps, buckles and the like may help secure the shell to the wearer. Increasing the strapping force would increase the corrective force in a limited manner but eventually a new orthosis would be needed to properly apply the desired spinal curvature. This is especially an issue with children who-in addition to the altered curvature of their spines due to the success of their treatment-also outgrow their orthoses. The present invention can be used to alter the forces applied across an orthosis to accommodate the changing needs of the wearer.

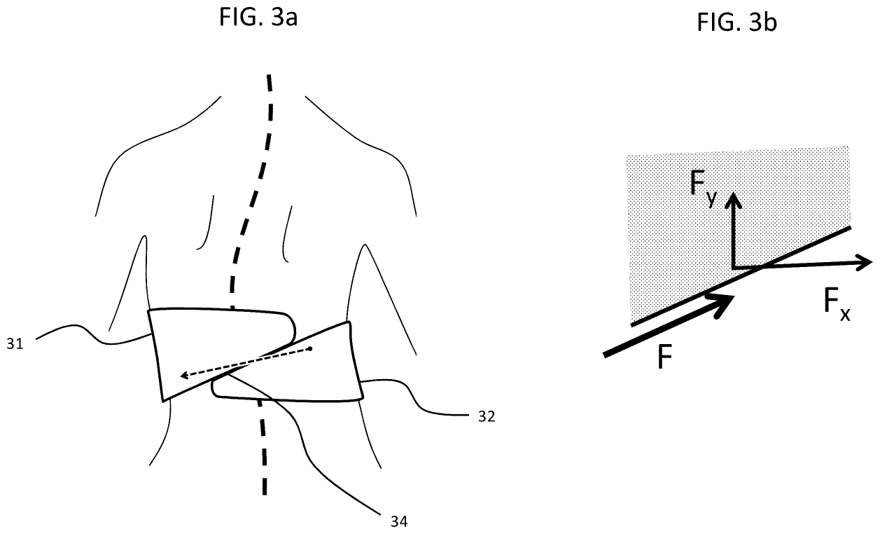

FIG. 3a illustrates an aspect of the present invention. Two body panels (31 and 32) wrap around the wearer's torso. The body panels may be selected from a choice of off the shelf sizes, fully customized for the wearer (e.g., a 3D printed article based on a scan of the patient's torso), or partially customized (e.g., an existing article that is heat formed to fit the patient better). The body panels can be slidably attached along an edge (34) via a dovetail, rail, guide, tongue and groove, and the like. A force (shown as the dotted line) is generated between the body panels. Methods for generating a suitable force include connecting the body panels with a flexible tensioning component, a spring, an elastic band, a flexible cable connected to an adjustable tension component, a strap and ratchet, and the like. It should be noted that the panels could themselves be a mostly inelastic or lightly elastic fabric and/or elastomer.

The force vector shown in FIG. 3b can be resolved in the x and y directions. This vector diagram shows that by applying a force between the body panels a force along the y-axis can be generated. This y-axis component of the force will impart a distraction force upon the vertebrae which lie in areas near and at the body panels. In a similar manner, a force along the x-axis can be generated. This x-axis component of the force will impart a translational force upon the vertebrae which lie in areas near and at the body panels. A distraction force is useful for traction orthotics; a translational force is useful for scoliosis orthotics. Changing the angle of the edge of the body panels (with respect to the transversal plane) and/or changing the location of anchor points of the mechanism that generates the force will alter the ratio of the amplitude of the x and y components of the applied force vector. In addition to elongating the spine, this force can also help tilt a segment of the spine in a scoliosis patient into a more favorable position.

In aspects, the lower body panel (32) rests on the wearer's hip and the upper body panel is fitted under the arm. The under-arm and pelvic bone make ideal pushing points from which to leverage joint distraction or alignment correction, and to ensure that the orthosis doesn't migrate from its desired position on the wearer's body. While not shown in a figure, the adjustable tensioning system can be configured to wedge under the arms and against the hips, and this tension can be adjustable by the user while wearing the brace to produce an elongating or distracting effect in the spine.

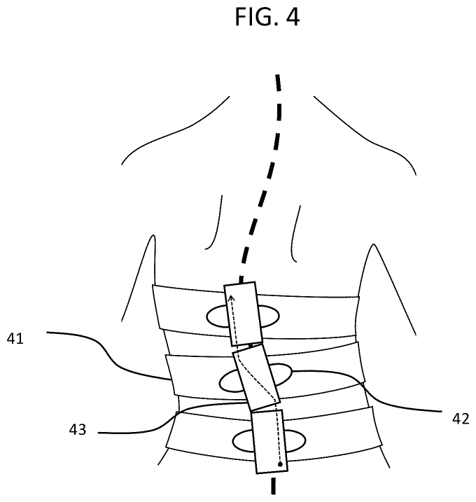

FIG. 4 shows another aspect of the present invention. A plurality of straps (41) are worn about the torso. The straps may be made of a semi-flexible material (e.g., neoprene) or rigid material. Preferably, the straps can conform to the wearer's torso for comfort, but be able to transmit a rotational and/or translation force to the body of the wearer without bunching or wrinkling. A rigid component (43) can be attached to the strap and is functionally connected to adjacent rigid component. An optional connection component (42) may be positioned between the rigid component and the strap. The connection component helps distribute the force applied by the rigid component over a broader area allowing the straps to be more flexible and still prevent bunching or wrinkling. A means for applying a force (not shown) between adjacent rigid components is provided, in aspects. The dotted line illustrated in FIG. 4 is an example of the path of a flexible tension component. A flexible tension component can be a flexible member that holds tension, for example a lace, cable, band, wire, or strap. One end of the flexible tension component can be anchored at the lowest rigid component. The other end of the flexible tension component can be connected to an adjustable tensioning mechanism anchored on the uppermost rigid component. Increasing the tension on the flexible tension component will rotate the rigid components to form a shorter path for the flexible tension component. As depicted in FIG. 4, the shorter path would induce rotations to the various rigid components which, when transmitted by the straps to the wearer's torso, would primarily have the effect of aligning the spine and reducing regions of compression within the spine.

In aspects, multiple rigid components may be attached to the same strap. In aspects, one or more straps may be connected to each other so that donning the orthotic is similar to pulling a shirt over the wearer's head. In aspects, the straps are connected to each other so that donning the orthotic is similar to donning a corset and tightening each strap individually.

If a flexible tension component is used to draw the rigid components together, the flexible tension component can be guided by a path, channel, tunnel, or the like inside the rigid component. In aspects, the flexible tension component can lie primarily on the outer surface of the rigid component and be guided by a channel, posts, rings or the like.

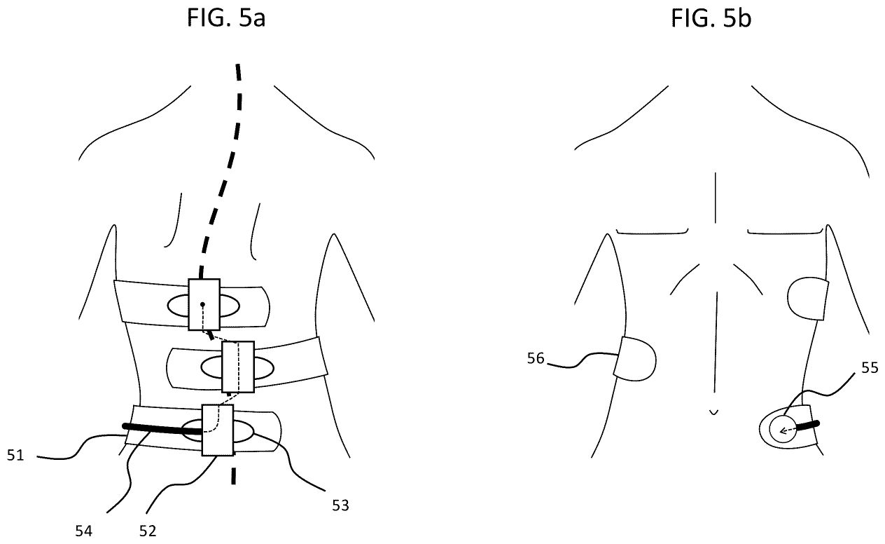

FIG. 5a illustrates another aspect of the present invention. A plurality of substantially rigid U-shaped body bands (51) hug the torso of the wearer as shown in the posterior view in FIG. 5a. In aspects, the U-shaped body bands do not completely encircle the torso but terminate at the front of the torso as shown in the anterior view of FIG. 5b. The body bands may be attached to a flexible substrate (not shown) (e.g., a shirt-like garment, torso sleeve, vest or the like) to make donning and doffing easier. A rigid component (52) can be attached to the body band. An optional connection component (52) may be positioned between the rigid component and the body band. The rigid components may be connected as in FIG. 4 or there may be a gap between them as shown in FIG. 5a. Alternatively a rigid component may be slidably connected to adjacent rigid components. In aspects, the rigid components are body panels.

One end of a flexible tension component (54) can be anchored at the topmost body band, traversing the rigid components sequentially, and the other end can be connected to an adjustable tensioning mechanism (also referred to herein as an adjustment mechanism or adjustable tensioning component) (55). As shown in FIG. 5b the adjustable tensioning mechanism can be positioned at or near the anterior side of the orthotic to enable the wearer ready-access. As depicted in FIG. 5a and FIG. 5b, adding tension to the flexible tensioning component (54) with the adjustable tensioning mechanism (55) would shorten the path between the rigid components. The shorter path would induce translation to the various rigid components which, when transmitted by the body bands to the wearer's torso, would primarily have the effect of straightening the spine.

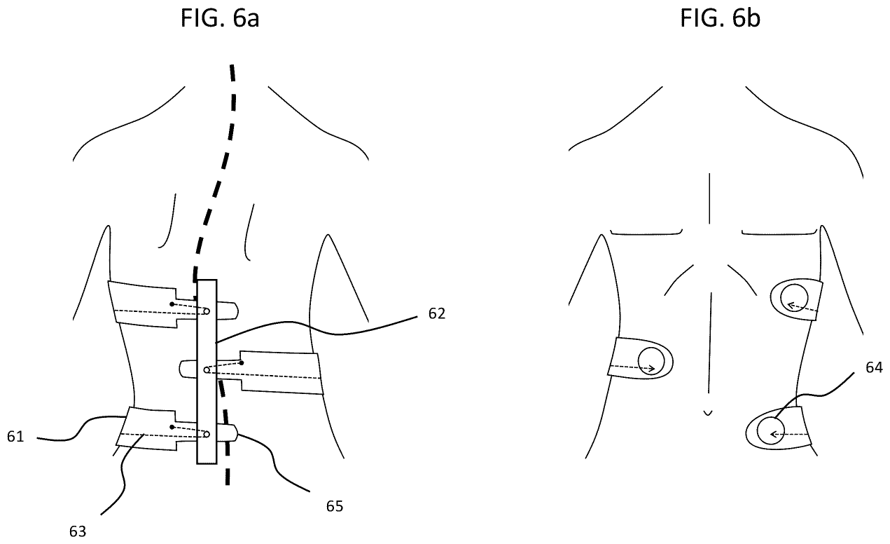

FIG. 6a and FIG. 6b show another aspect of the present invention. A plurality of substantially rigid body bands (61) are positioned on the wearer's torso. One end of the body band (65) can be slidably connected to a vertical beam (62). Suitable methods of connecting the body bands to the beam include, but are not limited to, tongue and groove, dovetail slides, rails, linear bearings, and the like. One end of a flexible tension component (63) can be anchored to the body band. The flexible tension component can loop back to a post or pivot on the vertical beam and return along the body band where the other end is connected to an adjustable tensioning component. As shown in FIG. 6a and FIG. 6b, in embodiments, each body band can have its own flexible tension component and adjustable tensioning mechanism so that each body band is able to move independently of the others. Adjusting the tension applied to the flexible tension component draws the body band to which it is attached deeper into the vertical beam. In aspects the vertical beam is composed of several connected vertical beams or a panel. The orientation of the slots, rails, etc. that connect the body bands to the vertical beam can be angled in different directions or positions. In this manner, the direction of the force that a body band imparts to the torso can be any body plane or combination of body planes shown in FIG. 1.

In another aspect, the vertical beam (63) and any or all of the plurality of body bands can be integrally fabricated where that connection point between the body band(s) and the vertical beam are able to move with respect to each other. The movement could be a translational (e.g. a sliding motion) with respect to each other (as needed in the embodiment shown in FIGS. 6a and 6b), but could also be rotational (as needed in the embodiment shown in FIGS. 5a and 5b) or a combination thereof. As shown in the various Figures, the body panels, body straps, body wraps, and the like are illustrated and as separate components for clarity. However, in aspects, the body panels, body straps, body wraps and the like may be fully or partially integrally combined but functionally separate. For example, the plurality of body bands and vertical beam of the embodiment of FIGS. 6a and 6b could be fabricated as a single physical element via 3D printing. The connection between the elements that function as body bands and the element that functions as the vertical beam needs to allow translation. This could be accomplished, for example, by making the connection(s) a zigzag or ribbon candy-like tortuous path. A flexible tension element anchored to one of the body band elements, looped around a pivot or post on the vertical beam element, and connected to an adjustable tensioning component anchored on the body band element would translate the body band element towards the vertical beam element.

As used in this document, the phrases body panel, body wrap, body band are synonymous and are used to describe functionally separate elements of the present invention. A functionally separate element is defined as an element, part, or component that can translate, rotate, move, or be positioned substantially independently from another functionally separate element. In aspects, functionally separate elements may be physically separate objects. In aspects, functionally separate elements may be integral with another functionally separate element or elements when the connection between the functionally separate elements allows for translation, rotation, or movement.

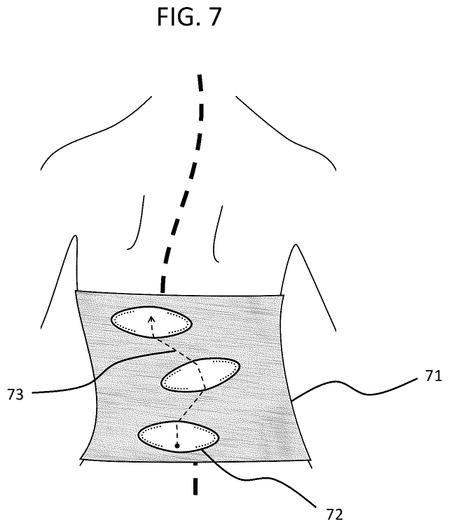

FIG. 7 shows another aspect of the present invention wherein substantially rigid panels (72) are sewn or otherwise attached or connected to a garment (71). A flexible tension component (not shown) connects the panels in sequence by running along the path depicted by the dashed line. One end of the flexible tension component can be attached to the bottom-most panel. The other end of the flexible tension component can be connected to an adjustable tensioning mechanism attached to the uppermost panel. Putting the flexible tension component in tension will rotate and translate the panels to shorten the path of the flexible tension component.

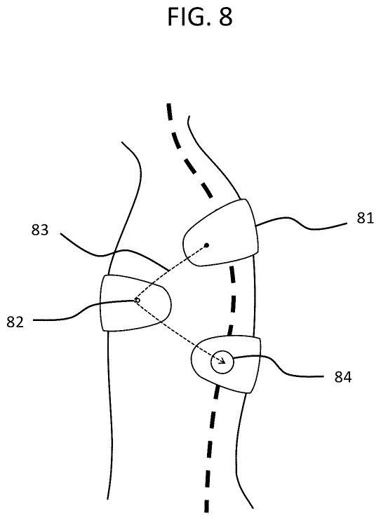

FIG. 8 is a lateral view of another aspect of the present invention. A plurality of substantially rigid body bands (81) can be positioned on a patient's torso. A flexible tension component (not shown) can follow the path depicted by the dashed line and connects the body bands sequentially. One end of the flexible tension component can be anchored at the topmost body band. In embodiments, the flexible tension component loops around a post or pivot on the middle body band and the other end terminates at an adjustable tensioning component (84) connected to the lowest body band. As illustrated, the anchor/pivot points can be primarily located laterally on the body bands. When a tension is applied to the flexible tension component, the anchor/pivot points are drawn towards the centerline straightening the spine.

In aspects a second flexible tension component and tension adjustment mechanism are used on the medial side of the body bands. Alternatively, the flexible tension component follows a guide from the lateral to the medial side of the topmost body band, continues to a pivot point on the medial side of the middle body band, and terminates at the medial side of the lowermost body band. The two aspects described in this paragraph help evenly distribute the forces applied to the body bands so that rotation about the long axis of the body (an axis normal to the transversal plane) is minimized and translation of the body bands is maximized.

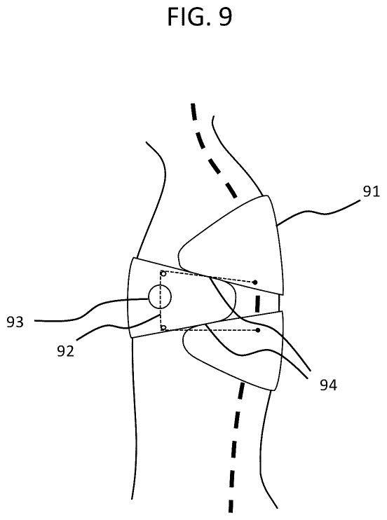

FIG. 9 illustrates a lateral view of three wedge shaped body panels (91) that are slidably attached along adjacent edges (94). A flexible tension component (not shown) follows the path depicted by the dotted line. One end of the flexible tension component can be connected to the uppermost body panel. The flexible tension component can be coupled to an adjustable tensioning component (93) and the other end of the flexible tension component can be connected to the lowermost body panel. Applying tension to the flexible tension component draws the body panels towards each other. The wedge shape of the body panels primarily imparts a distraction force to the wearer's spine.

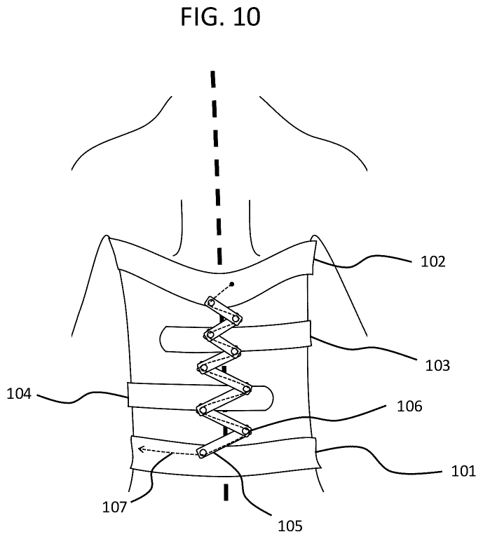

FIG. 10 is a posterior view of another aspect of the present invention. A substantially rigid body band (101) can be positioned on the hips of the wearer. A substantially rigid body band (102) can be positioned under the arms of the wearer. A plurality of support bands (103, 104) can be positioned between the upper and lower body bands. The support bands may be substantially rigid in which case they may only wrap around a portion of the torso as illustrated. Alternatively, the support bands may be semi-flexible straps in which case they would preferentially wrap completely around the torso. A plurality of rigid bars, also referred to as correction elements, (105) are linked at pivot points (106). A flexible tension component (not shown) follows the path depicted by the dotted line and threads through (or around) posts or pivots attached to the rigid bars or correction components. Applying tension to the flexible tension component will shorten its path thereby straightening the rigid bar linkage and creating a distraction force across the plurality of body bands, straps, or panels.

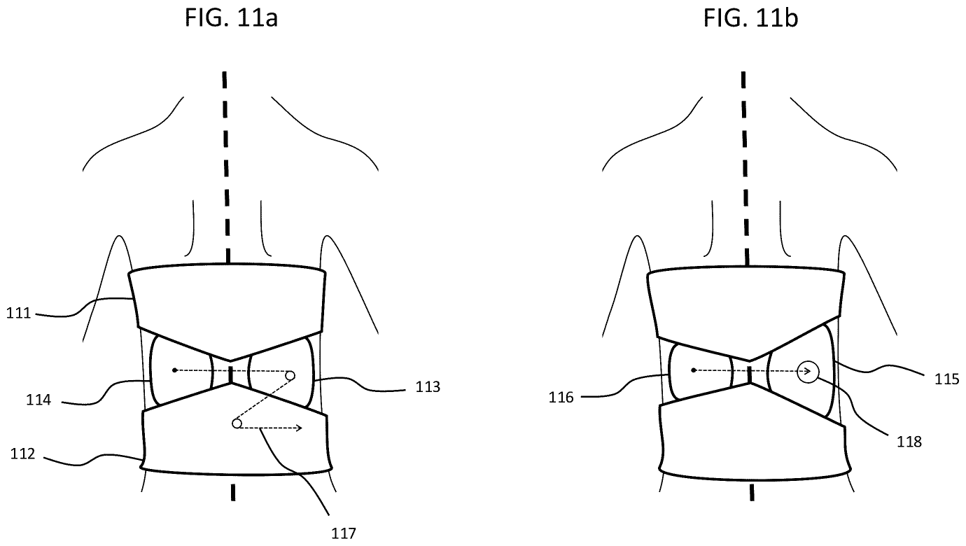

FIG. 11a and FIG. 11b show a posterior view of another aspect of the present invention. An upper body panel (111) can be positioned under the shoulders of the wearer. A lower body panel (112) can be positioned on the hips of the wearer. The lower edge of the upper body panel and the upper edge of the lower body panel can be V-shaped wherein the cusps of the V's point towards each other. A left wedge shaped panel (114) and a right wedged shaped panel (113) can be positioned between the upper and lower body panels. The upper edges of the wedge shaped panels can be slidably attached to the lower edge of the upper body panel. The lower edges of the wedge shaped panels can be slidably attached to the upper edge of the lower body panel. A flexible tension component (not shown) follows the path of the dotted line in FIG. 11a. One end of the flexible tension component is connected to the left wedge shaped body panel. The flexible tension component loops around a post or pivot on the right wedge shaped body panel and loops around a post or pivot located along the centerline of the lower body panel and continues along a path to the front of the lower body panel. The second end of the flexible tension component is attached to an adjustable tensioning component (not shown) attached to the front of the lower body panel. When a tension is applied to the flexible tension component the left and right wedge shaped body panels are drawn towards the centerline of the torso and generate a distraction force across the vertebrae of the wearer's spine.

FIG. 11b shows a similar mechanism, but whereas the wedge shaped body panels in FIG. 11a were more or less symmetrical, the left wedge shaped body panel (116) in FIG. 11b is differently shaped than the right wedge shaped body panel (117). A flexible tension component (not shown) follows the path depicted by the dotted line. One end of the flexible tension component can be anchored to the left wedge shaped body panel. The other end can be connected to an adjustable tensioning component (118) attached to the right wedge shaped body panel. Applying tension to the flexible tension component draws the right and left wedge shaped body panels together but because they are differently shaped, the distraction force is greater on one side than the other side. The mechanism in FIG. 11b creates a distraction force and a rotational force on the spine at the same time.

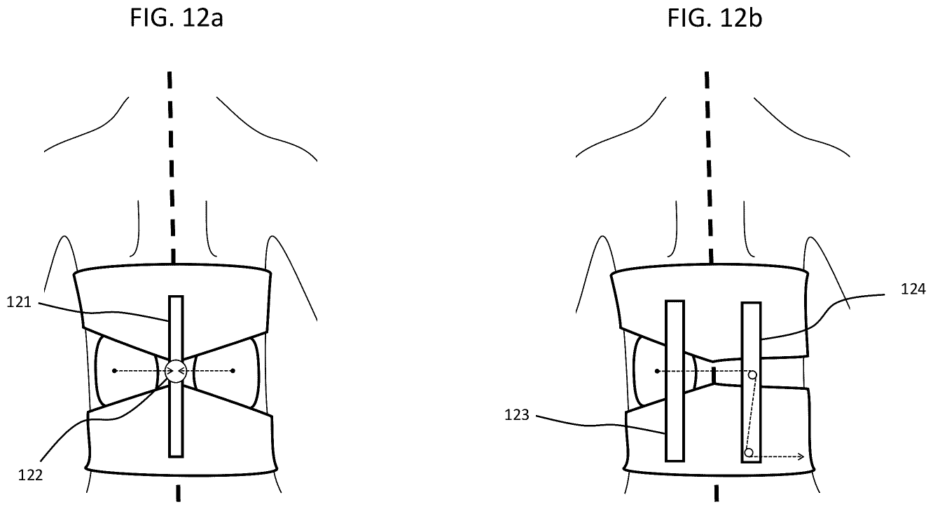

FIG. 12a and FIG. 12b are posterior views of another aspect of the present invention. Similar to the mechanism shown in FIG. 11a and FIG. 11b, the upper and lower body panels are connected to a rail (121). At least one of the connections between body panel and rail can be a slidable connection. Applying tension to the two flexible tension components (not shown) that follow the paths depicted by dotted lines by means of the adjustable tensioning component (122) draws the right and left wedge shaped body together which lifts the upper body panel away from the lower body panel to generate a distraction force. The addition of the rail helps stabilize the upper and lower body panels with respect to each other and prevent unwanted rotation along an axis normal to the coronal plane.

The mechanism shown in FIG. 12b uses two rails (123, 124) to connect the upper and lower body panels. A single wedged shaped panel is drawn towards the body centerline generating a distraction force. The double rail system ensures that the upper and lower body panels don't rotate along an axis normal to the coronal plane even though the wedging force is asymmetrical.

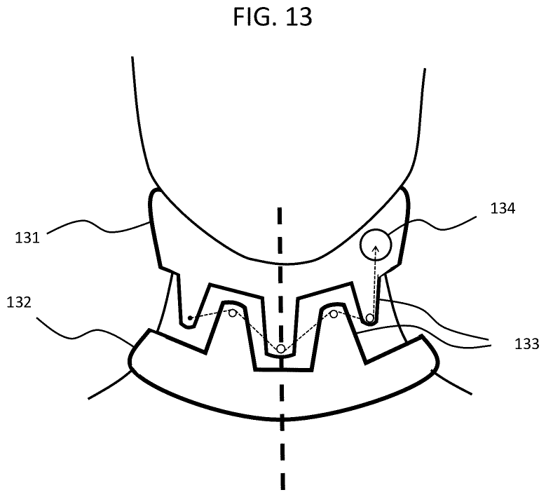

FIG. 13 shows an anterior view of another aspect of the present invention. The mechanism in FIG. 13 can be incorporated into a neck brace and provide a distraction force to the cervical vertebrae, but the same principle can be applied to a back brace. An upper neck rest (131) is positioned under the wearer's chin. A lower neck rest (132) is positioned above the wearer's shoulders. A plurality of fingers (133) extend from the lower edge of the upper neck rest and upper edge of the lower neck rest. The fingers of the upper and lower neck rests interlace with each other. A flexible tension component (not shown) follows the path depicted by the dotted line. One end of the flexible tension component can be attached to the far left finger. The flexible tension component zig zags between pivots or posts on the remaining fingers. The other end of the flexible tension component can be connected to an adjustable tensioning component that is attached to the upper neck rest. Applying a tension to the flexible tensioning component shortens the path of the flexible tensioning component pushing the upper and lower neck rests apart thereby imparting a distraction force to the cervical vertebrae.

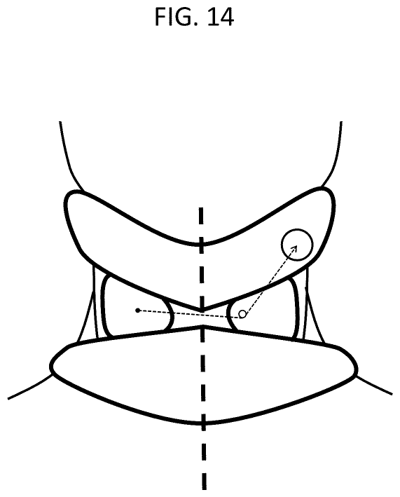

The neck distraction mechanism illustrated by the anterior view in FIG. 14 is similar to the mechanism of the back brace shown in FIG. 11b. Wedge shaped components that are slidably attached to upper and lower neck rests can be drawn towards the orthotic centerline and impart a distraction force to the cervical vertebrae.

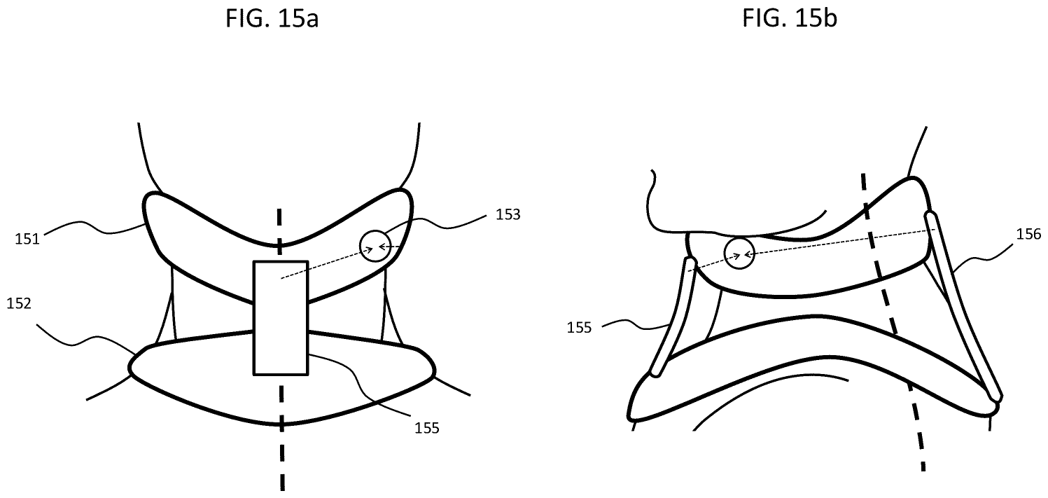

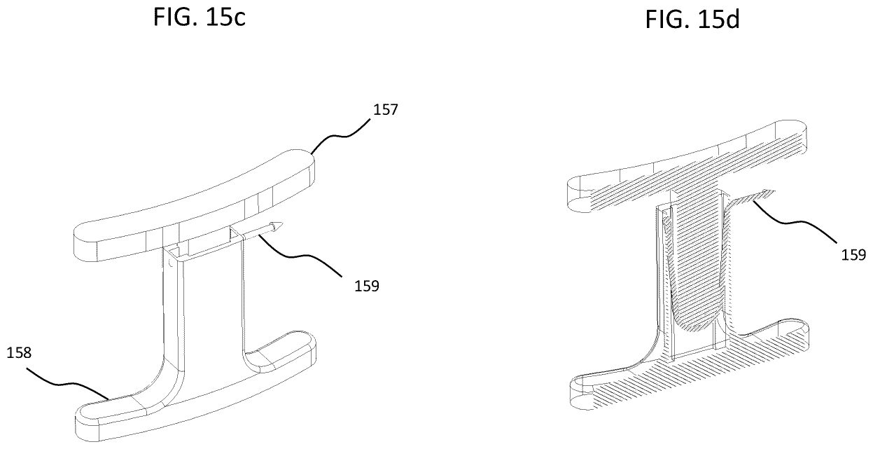

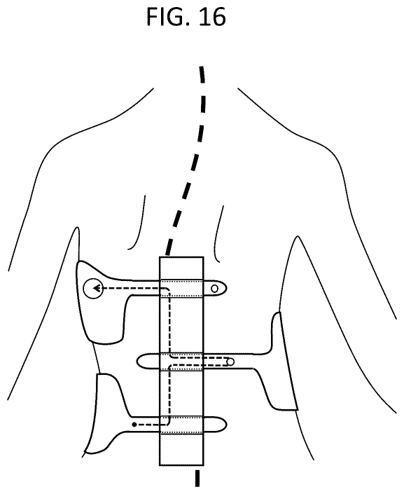

FIGS. 15a-15d show other aspects of the present invention. An upper neck rest (151) and a lower neck rest (152) are worn by the patient. Tongue (157) and groove (158) mechanisms can be incorporated into the anterior connection bar (155) and the posterior connection bar (156). In the anterior connection bar, a flexible tension component (159) can be anchored on one side of the groove. In embodiments, the flexible tension component travels down the groove and loops around the end of the tongue and then returns to the top of the groove where it exits. In embodiments, the flexible tension component continues to an adjustable tensioning component (153) where the second end is connected. In aspects, this embodiment can include a second flexible tension component similarly positioned in the posterior connection bar and connected to the adjustable tensioning component. Applying tension to the flexible tension components forces the tongues out of the groove thereby creating a distraction force across the cervical vertebrae. The magnitude of the distraction force can be increased or decreased with the adjustable tensioning component (153).

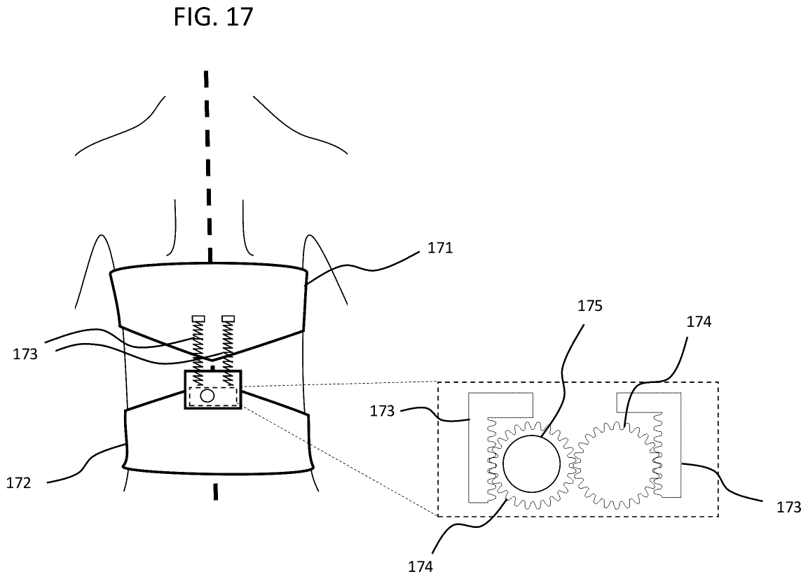

FIG. 16 shows how a similar mechanism can be employed to generate translational forces across the vertebrae in the spine, featuring a vertical beam and body panels connected slidably to the vertical beam on tracks or grooves. Shortening of the path of the flexible tensioning component upon tensioning (represented by the dotted line) causes the body panels to translate horizontally and inward to provide a three-point corrective force to the spine.

FIG. 17 illustrates another aspect of the present invention. An upper body panel (171) can be worn under the shoulders and a lower body panel (172) can be worn resting on the hips. The upper and lower body panels can be connected by a rack and pinion mechanism. Two racks (173) can be connected to the upper body panel and can be positioned side by side with their teeth facing towards each other. Two pinions (174) can be connected to the lower body panel. In embodiments, the pinions intermesh with each other and with the racks, as shown in the enlargement. A knob (175) can be connected to one of the pinions. Turning the knob clockwise drives both racks upwards, lifting the upper body panel away from the lower body panel thereby creating a distraction force across the vertebrae in the spine. This embodiment represents that the corrective and/or distraction forces described in the previous embodiments comprising flexible tensioning components can also be achieved with components under compression.

If the orientation of the rack and pinion is angled with respect to the vertical axis, a rotational force can be imparted in addition to translational forces.

In aspects, combinations of the various methods, mechanisms, embodiments, aspects, devices, components, etc., described herein can be used and/or combined together, in aspects, to optimize the clinically ideal or preferable treatment of rotations and/or translations of the spine. Likewise, the number of body bands, wraps, or panels may be increased or decreased compared to the numbers illustrated in the Figures.

In embodiments, the invention described herein uses a flexible tension component (or components) to adjust the magnitude of the translational and/or rotational forces applied across the vertebrae of the spine while the patient is wearing the orthotic. The direction of the translational and/or rotational forces are determined by the geometries incorporated into the orthosis. Using a flexible tension component (or components) can enable the many different mechanisms described above, which can be combined to readily customize a plurality of translational and/or rotational force vectors, which can be prescribed to produce a clinical effect (e.g., straighten the spine). An elastic component may be incorporated inline with the flexible tension component to provide a way to store energy.

In aspects, the path of the flexible tension component may be user-adjustable using, for example, anchors. In other aspects, additional adjustment mechanisms may be used, or switches may be employed, to generate a force or forces in different directions or combinations of directions as needed.

The anchor points may be determined to optimize function through algorithms, artificial intelligence, machine learning, and general software-automated design of the fabricated device. The anchor points or connection of the tensioning component may be modified and placed at any point or angle of the proximal or distal portion of the device. There may be unlimited orientations and positions across the surface of the device. Alternatively, the anchor points may be pre-set to limit options for force direction. For example, in a scenario, a patient with scoliosis is scanned, the path of the spine is mapped, corrective force direction and magnitude is calculated, and this is automatically built into a custom orthotic.

Additional aspects and embodiments of the present invention are described below.

In embodiments, the device described herein is an optionally user-adjustable corrective neck or spine orthosis, which can direct a force or forces around a region of the spine in one to three axes. Depending on the embodiment, the device is capable of generating both linear and rotational forces around the spine. Embodiments of the device can be custom fit, or custom fabricated to the user's morphology and clinical indication in various aspects. In embodiments, it can be adjusted by the user, manufacturer or clinician through multiple features including, but not limited to: 1) a tensioning or compression component/apparatus where the user can adjust the magnitude and direction of corrective force(s) based on their pain, activity, clinical indication, corrective protocol, and/or clinically recommended protocol, 2) optionally, a mechanism for changing the direction/orientation of corrective force or forces based on the positioning of specific pressure plates, or 3) optionally, a method to custom-fit or custom-fabricate the shape or form of the device to the user's anatomy to optimize or improve comfort and/or efficacy. In aspects, the device may be modular to extend along different regions of the spine. In other aspects, the device may comprise loosely coupled panels or flexible jointed regions in order to allow movement relative to the curvature of the user's body. Depending on the embodiment, the device may provide corrective forces to align the spine (e.g., correcting scoliosis), provide therapeutic benefit to unload or distract a targeted region of the spine, and/or offer post-operative and rehabilitative benefit by selectively immobilizing or unloading the joint.

In some embodiments, the back orthosis is designed to correct the alignment of the spine, for example to treat scoliosis. In other embodiments, it is designed to distract a region of the spine to relieve pain, support regeneration, or limit stenosis. In other embodiments, the device is therapeutic, designed to apply pressure to a region of the spine as do traditional back braces but in a user-adjustable manner. In other embodiments, the device may be used to limit motion in a specific axis of rotation for post-operative applications; in aspects, this is accomplished by applying different amounts of tension and/or compression around different regions of the back, the shoulders, the neck, and/or between the back and hips. Tension or compression components (generally referred to as energy storage components) can be incorporated to manipulate forces within the spine by applying leverage against specific anatomical features, such as the hips, torso, shoulders, or head. The applied tension (or compression) can be fixed, adjustable, or dynamically adjustable in that tension (or compression) increases with increasing flexion, extension, or rotation, of the spine in the sagittal, coronal, or horizontal plane, respectively. In other embodiments, the same directional force or forces may be applied to augment motion of the back or shoulders, for example while lifting an object. In this way, the embodiments described herein include what are traditionally described as passive and active exoskeletons. In this way, the device may act as a preventative measure for spinal injury or compression, in addition to its therapeutic applications. Through this mechanism, the brace allows mobility within certain axes while limiting it in others to maximize or improve the freedom of the joint while providing (only the) necessary directional support.

Some embodiments comprise one or more body plates or panels, which are rigid or semi-rigid portions designed to conform to the user and distribute a load, imparted by the tensioning system. The body plates may be connected directly by the flexible tensioning components or energy storage components. They may be loosely coupled with slots. In other embodiments, one body plate may have flexible joints, which include thinner regions of material allowing the plate to flex at the joint in order to better conform to the spine. In other embodiments, the device is comprised of a shirt, sleeve or other fabric component, throughout which the body plates are incorporated, and in aspects, allowing the tensioning system to be channeled or woven throughout. In this example, one of the body plates houses an adjustment mechanism, which may be a dial, to increase or decrease tension within the tensioning component, thereby imparting a force along the path of the cable path. The amount of tension may be tailored to an individual's needs, and/or it may be adjustable (such as by the wearer). The tensioning or compressive component may be external, or partially-or fully-integrated within the brace or sock.

The devices described herein may be customized to address the user's needs. This includes features, such as, by way of example, custom fabrication from a 3D-scan or measurements, custom fit through modification by a certified professional (e.g., thermoforming), adjusting the amount of force(s) applied in a given direction(s) using an adjustment mechanism connected to a tensioning component (e.g., containing elastic bands) or a compression component (e.g., containing springs), adding or removing modular components (including, for example, additional body plates), and/or changing of the direction in which force is applied using anchors or through custom fabrication, including the axis about which one or more moment is generated. Tensioning or compression components may be in the form of bands, sheets, or lines, and integrated within or external to the device. All of the reference components or components may be directly or indirectly connected or attached. The size and geometry of these components may be tailored to yield specific mechanical properties. Inelastic and elastic components may be combined in patterns or arrangements to control local forces in areas of the vertebrae or back where it is desired.

Mechanisms to Control the Direction and/or Magnitude of Force

In embodiments described herein, the device(s) allows for the user/wearer to adjust the force or forces generated by the device around their own body. While a medical professional may select or fabricate the device to set the bounds of adjustability, including magnitude and/or direction of force, the user can control the amount of force in incremental or continuous manners. The applied force imparts a dynamic functionality to the device, whether that be limitation of movement, augmentation of movement for rehabilitation, corrective function, and/or therapeutic effect. Among others, a unique aspect of the device relates to the ability to dynamically adjust the amount of tension in one or more directions around a region of the spine, described herein as a multi-axis rotation control device. The term multi-axis means that tension can be generated across at least one axis.

The amount of force generated in a given direction may be controlled dynamically or statically via energy storage component(s) in compression or tension. Energy storage components are described as any component or component that stores mechanical energy including, by way of example only, elastomeric bands, webs or other geometries, springs, pneumatic components, electromagnetic components, pneumatics, or any component that may act as a mechanical energy storage component that may be adjusted to hold variable forces. In aspects, the force within the component will be set by the user at a certain position, and will change throughout a range of motion.

For example, a tensioning component may run in the longitudinal direction through body plates along the spine. When tensioned, the components may increase the rigidity of the device to prevent flexion of the spine. This feature may be useful to limit a range of motion post-operatively, and to allow an increasing range of motion over time. In the same way, a compressive component (e.g., a spring) placed on the anterior side of the body may also limit or prevent inversion of the back to address the same indication. By adjusting the magnitude of force within the energy storage component, the user can prevent spinal compression, reduce pain, unload a region of the joint, or limit a range of motion.

The user can adjust the magnitude of force or forces within the tensioning or compression component using an adjustment mechanism, for example. An adjustment mechanism as described herein can allow the user/wearer of the device to increase, decrease, or remove, the amount of force stored within the tension or compression component and therefore the force or forces generated around the back joint. In aspects, the adjustment mechanism may comprise a dial, lever, ratchet and pawl system, pulley, electric motor, etc., to modify and/or control the amount of force or forces supplied. The amount of force or forces may be recommended by a prescriber or other professional and indicated on the device. The range of forces able to be applied by the mechanism may be prescribed by a professional, e.g., forces of 20-30 pounds may be achieved in the component through a construction or inclusion of certain components by the manufacturer or professional to support a spinal correction regimen in line with the individual's current state. In other words, the device may incorporate tensioning components of varying storage modulus, size, cross section, and/or material or mechanical properties, in general. By way of example, energy storage components of greater modulus may be used earlier in a recovery period to substantially limit a range of motion, while components of lower modulus may be used later in the process.

In aspects, the wearer may be able to rapidly and conveniently adjust the device during activity, while the device is being worn. For example, the user may engage tension in the device while working out, increase tension while performing heavy lifting, and disengage tension while seated, within a span of seconds, in aspects. The tensioning/compression system may also have preset values or ranges. The tensioning/compression system may be adjusted manually or automatically via a system of at least one sensor and motor or actuator.

The adjustment mechanism may provide a range of mechanical advantage depending on the design selected for the specific use case. For example, an adjustment mechanism providing a mechanical advantage of 2:1 may be used in conjunction with tensioning components of a lower spring constant and for applications in which a lower amount of force is required to correct the back. In other examples, an adjustment mechanism with a 12:1 mechanical advantage may be selected in conjunction with higher durometer elastomers or for a high-torque application of the tensioning device, such as stabilizing the back during activity. A range of mechanical advantage from 2:1 to 24:1, by example, may be achieved in a dial-based adjustment mechanism. This mechanical advantage may be achieved in a compact design with a gear system which may include planetary gear systems, worm gears, or cycloidal gears and combinations thereof. Alternatively, pulley systems incorporated into the device, including within or between the body plates, can be included to provide a mechanical advantage upon adjustment.

In aspects, distraction systems are described. Distraction systems are defined as systems where a rigid, semi-rigid, or compressible movable component is located between and connected to a first functionally separate body panel element and a second functionally separate body panel element. When the movable component is translated or rotated, the distance between the functionally separate body panel elements increases, thereby imparting a distraction force between them (or a force causing separation). Examples of distraction systems include wedge shaped panels (116) and ramps (as shown in FIG. 11), a plurality of fingers (113) connected by a flexible tensioning component (as shown in FIG. 13), a tongue (157) and groove (158) mechanism (as shown in FIG. 15), and rack (173) and pinion (174) systems (as shown in FIG. 17). In the distraction systems described, the translation or rotation of the movable component may be controlled by an adjustable tensioning or compression system, which is operated by the wearer.

An orthotic that creates a distraction force using a motor, pneumatics, hydraulics or other means of transferring stored energy into motion is possible. The energy store could be a battery, compressed gases, chemical reactants, or the like. Such a device would be useful for patients who have limited mobility or strength. By pressing a button, opening a valve, etc. energy is transferred from the energy storage device to the energy conversion device which then imparts translational motion between one body plate and at least another body plate. An optional sensor that monitors the translational distance or force could be used to limit/adjust the amount of translation to a predetermined acceptable range.

An orthotic for decompressing the spine comprises a lower body plate that is secured to the wearer's hips and an upper body plate that wedges under the wearer's arms. (Alternatively, the lower body plate could rest primarily on the wearer's waist or upper chest and/or the upper body plate could primarily rest on top of the wearer's shoulders or under the wearer's chin.) The upper and lower body plates are connected by at least one slidable connection component. At least one linear actuator where one end is secured to the lower body plate and the other end is secured to the upper body plate is provided. There are many methods of driving and energizing a linear actuator but a common method is to use an electric motor to rotate a worm which engages with a worm wheel to drive a screw. A nut coupled with the screw translates as the screw turns. The advantage of a worm gear transmission is that worm gears cannot be backdriven. In a distraction orthotic, this means that the upper and lower body plates can be translated into position under power with a worm gear driven linear actuator, but will remain in position if the power is disconnected.

The adjustment mechanism may be used by the wearer/user to optimize or improve usability for a certain activity, to be used at a certain stage of rehabilitation, or be based on the current medical condition (e.g., if the user has a greater degree of pain on that day, they may add more tension to the component to further decompress the spine). The degree of force or forces generated or stored within the tensioning or compression component may be modified based on a user-controlled dial, allowing the user to change the force at any increment between zero to a maximum force based on activity. The user can adjust a force or forces, in embodiments, while the user is wearing the device. The degree of unloading may be modified by interchangeable components, e.g., springs or bands of different spring constants. In addition to being able to adjust tensile force from zero to a maximum force continuously or in increments to achieve any force within the range, the device may allow for tensioning to specific discrete forces (e.g., by way of example only, in 0.1 lb., 0.2 lb., 0.3 lb., 0.4 lb., 0.5 lb., 0.6 lb., 0.7 lb., 0.8 lb., 0.9 lb., 1.0 lb., 1.1 lb., etc., and so on, increments), or may simply toggle between on and off (e.g., minimum (or zero) and maximum force). The force or forces may be adjusted in any number of increments including on or off, stepwise, stepwise between a minimum and maximum force, or continuously on a gradient.

The tensioning (or compression) components may be constructed of different materials to provide different properties based on the user's need. For example, a tensioning component consisting of an elastomeric component of certain viscoelastic properties may better mimic the feel of natural muscle, tendon, and ligament complexes, while a more rigid or fully rigid tensioning component may completely lock the back in one or more directions or prevent movement beyond a specific range of motion.

In order to control forces and/or rotation around one or more axes, the device may contain multiple tensioning or compression components to address the user's need. In aspects, one or more tensioning or compression components may be controlled by a single adjustment mechanism. The tensioning components can be modular and added as needed based on the user's condition.

Additionally, the device can contain one or more adjustment mechanisms of the same or varying design (e.g., rotary dials, levers, ratchet and pawl mechanisms, or motors) that change the magnitude of force or forces within the tensioning or compression component(s) around one or more axes.

The adjustment mechanisms may also change the direction of force by moving anchor points, changing tensioning or compression component(s) geometry, or changing orientation of the tensioning or compression component(s).

### Custom Fabrication and Custom Fit

The device may be further customized through a user-specific fit. The shape of any of the body plates or tensioning or compression component may conform to the user's body. A 3D scan may be used to manually, automatically, or semi-automatically fabricate a custom device, for example through 3D printing, or additive manufacturing, or subtractive manufacturing. In aspects, the device may consist of both custom and non-custom components. The custom fit, in aspects, will help ensure, optimize, or improve, user comfort and maximize or improve effectiveness by controlling contact area and resulting force(s) distribution where the device is affixed to the user. For example, the upper portion of a decompression brace may be designed to distribute force around the user's torso and shoulders, while a lower portion conforms to the user's hips. Custom design and fabrication may not just determine personalized fit, but also the direction of force across and/or between the joint. For example, anchor points or channels in the device to guide the tensioning (or compression) component may be automatically or semi-automatically positioned in the device based on user need, prescription, gait analysis, or other data. The overall shape of each body plate along with the path of the tensioning (or compression) system and connecting components, may be automatically designed based on scan data, user morphology, radiographic data, prescription data, and/or user-provided data (e.g., pain within a region of the joint), to automatically adjust forces around and/or within the spine. This may be demonstrated on a digital rendering of a 3D model of the user to understand the corrective forces and expected progression of spinal correction during treatment. Additionally, the adjustment mechanism may be placed using similar data and automated design processes.

The device may be prefabricated and can be further modified by the user or a professional to provide the desired function. Modification may include molding or thermoforming of the rigid components of the device to improve fit, comfort and/or function. Modifications may further include adjustment of anchor points to create the intended correction or unloading force or forces in the individual. The device's modular design can allow a certified fitter or doctor to modify the device's performance based on the specific user need. Sections of the body plates may be trimmed to fit. Additionally, the device may be constructed from modular segments depending on the location of the spine for targeted therapeutics. For example, two or more modular components may be combined, including the incorporated tensioning (or compression) component, to address the lumbar (L1-L5) and thoracic (L1-L12) regions.

The device may be connected to and/or used in conjunction with additional orthopedic or prosthetic devices using universal or custom connectors. The device may be modular for compatibility with different devices based on the connection required. Other devices which may be connected to the device include, but are not limited to, hip orthoses, shoulder orthoses, neck orthoses, elbow orthoses, knee orthoses, or combinations thereof. Combination(s) of multiple devices with tensioning (or compression) systems may yield an effective, passive exoskeleton to prevent injury or improve productivity of the user. The device may also be continuously fabricated with such other devices. Any one of these orthoses may contain a similar adjustable tensioning mechanism, which can be used individually or adjusted with a single universally connected adjustment mechanism. In aspects, the combination of an adjustment mechanism, a flexible tensioning (or compression) component, and optionally an energy storage component, is referred to as a tensioning system or an adjustable tensioning system.

### Hinges and Track System

In embodiments, the body plates may be joined by hinges, brackets or track systems. When joined by hinges, the coupled body plates may be able to rotate relative to each other to allow rotation in the coronal plane, sagittal plane, and/or horizontal plane, depending on the hinge type and orientation. The hinges may incorporate inserts, e.g. pegs, to limit the range of motion in a specific direction while allowing range of motion in other directions. Brackets or track systems may be used to fasten and align body plates to yield a user-specific assembly to direct applied forces. For example, a scoliosis brace with three body plates, an upper shoulder plate, a midsection plate, and a hip plate, may be joined by a vertical bracket with slots or holes. In embodiments, each body plate, containing a series of holes, can be connected to the vertical bracket using fasteners to extend the device, increase the device width, and/or add additional body plates in order to address the size (or other needs) of the user. This feature can be beneficial in the application of scoliosis bracing, as the device may need adjustment every six or so months as the user grows and/or as the correction protocol progresses. The bracket may be a track, which contains slotted paths in which the body plates can be connected, but allowing the body plates to slide relative to one another (such as vertically) to allow movement within the longitudinal direction. In aspects, the track and/or body plates can comprise wheels, bearings, or lubricants. The slots may be designed or contain inserts to limit the distance over which movement is allowed. The bracket or track may be combined with additional components in the horizontal direction (or other directions, such as vertical and/or diagonal). Depending on the fastening, the body plates may individually rotate or translate within a discrete range to accommodate a limited or complete range of motion depending on the application. Tensioning or compression components may be fastened along the track paths or around hinges connecting body plates in order to generate local rotation or translation of an component(s) about a desired axis. In this way, the combination of the body plates, tensioning system(s), and hinges, braces or tracks (referred to collectively as fastening mechanisms), may be combined to provide targeted and user-specific forces throughout region(s) of the back and neck.

### Motors and Sensors

The tensioning or compressive components may be directly or indirectly connected to a motor to control the increase or decrease in force between, around, and/or within the body plates. The motor may be controlled by an electronic interface connected directly or indirectly to the device. The interface may be an app (computer-implemented application), processor, and/or software, on the user's mobile device, smart watch, or other computer/smart device/CPU. In aspects, the device may comprise one or more processors, one or more motors, one or more controllers, one or more sensors, one or more antennas, as well as software to control, instruct, process, command, and/or implement, adjustment of the tensioning (or compression) component(s) of the device. The motor and/or controller components may control or change force or forces within the tensioning or compression component(s) automatically based on input from sensors located on the device or elsewhere on the user. For example, EMG sensors and/or accelerometers may detect a certain flexion or movement of the back throughout a range of motion. Pressure sensors incorporated with body plates, or tensiometers incorporated into the tensioning component, may measure the amount of force or forces generated by the device for corrective application. These sensors may communicate with a mobile application, which advises the user on the amount of tension (or compression) to use or an adjustment to the position of a body plate, in order to achieve a desired and/or prescribed corrective force or forces. Sensors which determine the relative position of the body plates or movement/alignment of the body plates may also yield data directly or indirectly related to spinal position. This information may be combined with the patient's original clinical and radiographic data, in combination with theoretical or a database of information, to monitor and optimize/improve the corrective forces subjected on the spine. This data-based approach allows the user to follow a clinical protocol (e.g., subjecting the spine to a target range of corrective force or forces in target location(s) for a specific set of time(s)) in order to, in aspects, reduce the overall time required for the corrective procedure (e.g., daily), or allow the user control over the times at which the corrective force or forces are applied. This is a significant advancement, as current scoliosis braces, such as the Boston™ brace, are required to be worn for 18 hours but are static and provide a limited range of corrective force. In other aspects, sensors worn as part of the device or independently from the device may measure biometric information and movement, for example breathing patterns of the user. This data can be relevant for the corrective function of a device, as existing back braces like the Chenaeu™ brace rely on breathing to generate corrective force.

In another example, sensors may trigger the energy storage components to engage while the user is bending over, in order to support the back during strenuous activity. In the case of application as an active exoskeleton, sensors may detect load or allow user input of a desired task through, by way of example only, an interface, a mobile interface, and/or voice command, in order for the device to support the specific strenuous activity. In other aspects, the tensioning (or compression) component can be manually adjusted using, for example, a smartphone, smart wearable technology, computer, external processor, or an electronic or mechanical control mechanism on or attached to the device (or in any communication with the device). These adjustments can be made in real-time or substantially real-time as understood by one of skill in the art; they can be made by the user or a treating practitioner; and/or they can be made while the user is wearing the device. A combination of both automatic and manual adjustment is also contemplated. Part or all of the system described herein can be combined with an exoskeleton (passive or powered) and used to help a patient stay upright and/or ambulate.

In other aspects, the brace may automatically adjust during certain times of a day, activity, or position. For example, it may be easier and more comfortable to correct the spine while the user is in a supine position, for example during sleep, at which time the brace may be activated or remind the user via a notification to activate/adjust the device before sleeping.

In aspects, sensors such as a Fuji pressure sensitive film can be incorporated into the body plates so that the amount of corrective or distraction forces can be measured and accurately maintained by the adjustable tensioning component.

### Method of Making

The various embodiments of the present disclosure may use traditional manufacturing processes for back braces and/or 3D printing/additive manufacturing to produce the components and/or the overall device. These techniques may also be used to fabricate positives through which negative molds are constructed for injection molding.

The fabrication technique can allow for low-cost, custom devices. It also can enable manufacturing of intricate parts containing internal channels and features that could not be feasibly or affordably produced with injection molding, machining, or other traditional methods of manufacturing orthotic devices. 3D printing enables efficient production of lightweight, yet durable materials such as thermoplastics. The result is a highly effective, lightweight, customized, and cost-effective device that can be manufactured at scale.

### Other Aspects of the Device

In aspects, the device comprises a series of interlocking plates (like the snake hinge). When disengaged, plates and the device can allow flexibility of the spine. When tensioned, they can interlock and support the spine in a rigid conformation. In aspects, the plates may conform custom to the back/chest/spine when interlocked to provide a stable conformation.

In aspects, the device comprises compression components located between body plates to generate a distraction force (in embodiments with body plates anchored above the hip and below the shoulder for spinal distraction or above shoulder and below head for cervical distraction).

In aspects, the device applies tensioning and or compressive components (e.g., anchored above the shoulder and above or around the hip) in combination.

In aspects, the interlocking components may allow movement in one direction (bending forward) but not another (bending back) to provide posture correction, support, and/or prevent injury.

In aspects the brace comprises a main belt that partially or completely circumferentially wraps around the torso. The main belt can be a rigid or semi rigid material that can be opened or closed via a closure system. The main belt can be adjusted to fit the user's size by overlapping ends that attach to one another as the closure system. In some aspects, this closure system is created via velcro, glue, magnets, or a latch.

In aspects, the main belt contains lateral plates that are rigid or semi rigid. The lateral plates can be inserted or removed from the belt as desired.

In aspects, the device comprises a posterior plate nested within the space of a posterior frame. The posterior frame can be rigid or semi rigid but acts as a supporting structure to link the posterior plate with the tensioning component. When tensioned, the posterior plate will move perpendicularly to the plane of the posterior frame. In aspects the posterior frame will include a guide rail that the posterior plate rests upon. The guide rail can serve as a stabilizing mechanism to ensure the plate moves in the intended plane.

In aspects, the tensioning component can include one or more of lace, elastics, or springs, to move the posterior plate. The tension can be customized by the user to the desired level of tightness or otherwise. In aspects, the tensioning component can include a sensor to display the level of force or forces output from the device. In aspects this output will be automatically released in a controlled manner over a designated period of time.

In aspects, the tensioning component will be controlled by one or multiple rotary devices, or pull tabs. These tensioning components, in aspects, increase or decrease the tension in a range from 0 lbs to 200 lbs. The increased tension can correspond to the perpendicular movement of the posterior plate.

In aspects, the tensioning component can be attached to the posterior plate in an asymmetrical manner. This can allow for the posterior plate to move perpendicularly to the posterior frame, as well as rotate about its axis, applying an uneven distribution of forces to the back.

In aspects, the user may be scanned using a mobile app. The app may incorporate algorithms, allow for a digital user interface, or apply machine learning/artificial intelligence to identify the current path of the back in different posture stages and identify an optimal position for the spine, as well as a series of corrective positions over time to adjust the spine into the proper condition (e.g., as a scoliosis brace is intended to work).

In aspects, the devices described herein can be combined with features of braces that are currently on the market, such as a pull-type fastening system that tightens the brace around the patient's waist, neck, or body, like traditional lumbar support brace. A pulley-type system can be used to increase the mechanical advantage when pulling the tensioning springs with one or more drawstrings. Such a system can also be used in conjunction with a ramping system proposed herein, so that the ramps create a corrective, distracting, or unloading force while simultaneously tightening around the patient's body producing compression. This mechanism may be applied to generate a force in the anterior direction to the lumbar region as the tensioning system is adjusted.

In aspects, the device's supporting software may provide for a custom-fabricated design of plates or an overall device configuration and instructions for assembly. It may provide for modular pieces to be subbed in/out at different time points. It may further provide for different adjustments to be made to the device at different time points in order to provide a corrective regiment for scoliosis. In aspects, a similar software-based method may be applied to provide a staged rehabilitative protocol for post-operative recovery/rehab or strengthening of the back. In aspects, the device incorporates electrical stimulation units for muscle activation and recovery. In aspects, the device incorporates electronic heating or cooling capabilities.

While the embodiments of the device described herein are external devices for exerting force on the spine through soft tissue by example, it is envisioned that these devices could be adapted to and integrated within the body as implants for the purpose of distraction or alignment of the vertebrae.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to, e.g., “some embodiments,” “an embodiment,” “one embodiment,” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A back orthosis comprising:
two or more rigid, semi-rigid, or flexible functionally separate body panel elements contacting a body of a wearer;
at least one flexible tensioning component that is connected directly or indirectly to the first functionally separate body panel element, the second functionally separate body panel element, and a tensioning adjustment mechanism;
wherein when a tension force on the at least one flexible tensioning component is increased, a position or positions of the first functionally separate body panel element and the second functionally separate body panel element move rotationally, translationally, or both, with respect to each other to provide a force or forces to a spine of the wearer.

2. The back orthosis of claim 1, wherein the first functionally separate body panel element and the second functionally separate body panel element are slidably attached along at least one edge of one or both of the first functionally separate body panel element and the second functionally separate body panel element.

3. The back orthosis of claim 1, further comprising at least one rigid or semi-rigid functionally separate vertical beam element, wherein the first functionally separate body panel element and the second functionally separate body panel element are slidably connected to the functionally separate vertical beam element on one or more tracks, and wherein when the tension force is increased, the first functionally separate body panel element and the second functionally separate body panel element translate towards a midline of the body of the wearer.

4. The back orthosis of claim 1, further comprising a main body which wraps around a waist of the wearer.

5. The back orthosis of claim 3, wherein the functionally separate vertical beam element is comprised of more than one segment, and wherein the at least one flexible tensioning component is connected to each segment of the more than one segment of the functionally separate vertical beam element.

6. The back orthosis of claim 1, further comprising two or more functionally separate body wrapping elements, wherein the two or more functionally separate body wrapping elements secure the two or more functionally separate body plate elements to a torso of the wearer.

7. The back orthosis of claim 1, wherein the tensioning adjustment mechanism is a dial, a pull tab, a lever, a ratchet and pawl system, a pulley, an electric motor, or combinations thereof.

8. The back orthosis of claim 1, further comprising an energy storage component, wherein the energy storage component is connected in line with the at least one flexible tensioning component.

9. The back orthosis of claim 8, wherein the energy storage component is a tensioning component comprising an elastomer, spring, or a hydraulic, magnetic, electromagnetic, or pneumatic mechanism.

10. The back orthosis of claim 1, further comprising one or more moveable anchor or anchor points attached to at least one of the two or more functionally separate body panel elements, wherein the one or more moveable anchor or anchor points direct a force or forces generated by the at least one flexible tensioning component across and/or around one or more axes of rotation of a wearer's back joint.

11. The back orthosis of claim 1, further comprising a garment to be worn by the wearer, wherein the two or more functionally separate body plate elements are connected to the garment.

12. The back orthosis of claim 1, further comprising one or more hinge, bracket, or track system, wherein the first functionally separate body panel element and the second functionally separate body panel element are connected by the one or more hinge, bracket, or track system.

13. A back orthosis comprising:
a first functionally separate body plate element with at least a first anchor point; and
a second functionally separate body plate element with at least a second anchor point;
wherein the first anchor point of the first functionally separate body plate element is laterally and vertically offset with respect to a wearer's spine from the second anchor point of the second functionally separate body plate element;
wherein an adjustable tensioning mechanism connects the first anchor point of the first functionally separate body plate element to the second anchor point of the second functionally separate body plate element;
wherein the adjustable tensioning mechanism comprises a flexible tension component and a tension adjustment component;
wherein tensile forces applied by the adjustable tensioning mechanism draw the first anchor point of the first functionally separate body plate element towards the second anchor point of the second functionally separate body plate element, thereby straightening a wearer's spine.

14. The back orthosis of claim 1, further comprising a plurality of correction components; wherein the correction components are in operational contact and connected by the adjustable tensioning mechanism; wherein the adjustable tensioning mechanism comprises the flexible tensioning component, wherein the plurality of correction components are connected to the two or more functionally separate body plate elements; wherein the plurality of correction components house the at least one flexible tensioning component in a tube or channel, wherein tensioning forces applied by the adjustable tensioning mechanism reduce a path length of the at least one flexible tensioning component between the tubes or channels of adjacent correction components thereby applying a corrective or distracting force or forces to a spine of the wearer.

15. The back orthosis of claim 14, wherein the plurality of correction components are pivotally connected.

16. A spine orthosis comprising:
a rigid or semi-rigid upper functionally separate body plate element secured to a first anatomical landmark of a wearer;
a rigid or semi-rigid lower functionally separate body plate element secured to a second anatomical landmark of the wearer,
a first track or groove located on the upper functionally separate body plate element and a second track or groove located on the lower functionally separate body plate element;
at least one wedge shaped panel located between the upper functionally separate body plate element and the lower functionally separate body plate element;
at least one flexible tensioning component connected on a first end to the at least one wedge shaped panel; and
at least one adjustment mechanism connected to a second end of the at least one flexible tensioning component;
wherein the at least one wedge shaped panel is movably connected to the upper functionally separate body plate element and the lower functionally separate body plate element by the first track or groove, the second track and groove, or both;
wherein by adjusting a tension within the at least one flexible tensioning component with the at least one adjustment mechanism, the at least one wedge shaped panel is translated towards a midline of a wearer's body; and
wherein a distance between the upper functionally separate body panel element and the lower functionally separate body panel elements increases as a result of adjusting the tension within the at least one flexible tensioning component with the at least one adjustment mechanism.

17. The spine orthosis of claim 16, wherein the spine orthosis is a cervical or neck orthosis.

18. A spine orthosis comprising:
a rigid or semi-rigid functionally separate upper body plate element secured to a first anatomical landmark of the wearer;
a rigid or semi-rigid lower functionally separate body plate element secured to a second anatomical landmark of the wearer;
at least one flexible tension component;
at least one tensioning adjustment mechanism; and
at least one channel or guide that enables the first and second body plate elements to move toward or away from each other;
wherein the at least one flexible tension component is connected on a first end to the at least one tensioning adjustment mechanism and on a second end to at least one of the upper functionally separate body plate element or the lower functionally separate body plate element; and
wherein by applying tension to the at least one flexible tension component, at least part of the first body plate element is forced away from the second body plate element, thereby creating a force or forces that separates the upper functionally separate body plate element from the lower functionally separate body plate element.

19. The back orthosis of claim 1, wherein the two or more rigid, semi-rigid, or flexible functionally separate body panel elements are custom fabricated to match a morphology of the body of the wearer.

20. The back orthosis of claim 1, wherein the two or more rigid, semi-rigid, or flexible functionally separate body panel elements are custom fabricated to correct a wearer's anatomy.

21. The spine orthosis of claim 18, wherein the spine orthosis is a cervical orthosis for a correction of, an alignment of, or a distraction of, a wearer's neck.

22. The spine orthosis of claim 18, further comprising one or more tongue and groove mechanisms, wherein the one or more tongue and groove mechanisms are positioned on opposite sides of a wearer's spine, wherein each tongue and groove mechanism translates approximately equally upon applying tension to the at least one flexible tensioning component, wherein a net force or forces applied by the tongue and groove mechanisms in combination to separate the upper functionally separate body plate element from the lower functionally separate body plate element is along a vertical axis of a wearer's body.

23. The back orthosis of claim 1, wherein the two or more rigid, semi-rigid, or flexible functionally separate body panel elements, the at least one flexible tensioning component, and the tensioning adjustment mechanism, are supplied as a kit for assembly by a certified professional or the wearer to meet one or more clinical needs of the wearer.

24. The spine orthosis of claim 18, further comprising an energy storage component, wherein the energy storage component is connected in line with the at least one flexible tensioning component.

25. The back orthosis of claim 18, further comprising an energy storage component, wherein the energy storage component is located between the upper functionally separate body plate element and the lower functionally separate body plate element.

26. The back orthosis of claim 1, further comprising one or more sensors, wherein the one or more sensors are capable of measuring a tensile force, a pressure, a compressive force, a position, an angle, or an acceleration corresponding to the two or more rigid, semi-rigid, or flexible functionally separate body panel elements, the at least one flexible tensioning component, the tensioning adjustment mechanism, or combinations thereof.

27. The back orthosis of claim 1, further comprising one or more modular components; wherein the modular components can be added, subtracted, or assembled in various orientations based on a clinical need of the wearer.

28. The back orthosis of claim 1, further comprising one or more compliant materials, wherein the compliant materials form to the body of the wearer when compressed to improve a comfort, a fit, and/or a function, of the back orthosis.

29. The spine orthosis of claim 18, wherein an energy conversion device converts energy into translational motion, rotational motion, or both, between the first functionally separate body plate element and the second functionally separate body plate element, to apply a distraction force between at least two vertebrae of a wearer's spine.

30. The spine and back orthosis of claim 29, wherein the energy conversion device drives at least one linear actuator to generate translational motion, rotational motion, or both, between the first functionally separate body plate element and the second functionally separate body plate element, to apply a distraction force across at least two vertebrae of a wearer's spine.
